(19) United States
(12) Patent Application Publication
(10) Pub. No.: US 2013/0061517 A1
(43) Pub. Date: Mar. 14, 2013

(54) Method for Growing Microalgae from Wastewater for Oil Production

(71) Applicant: David A. Hazlebeck, El Cajon, CA (US)

(72) Inventor: David A. Hazlebeck, El Cajon, CA (US)

(21) Appl. No.: 13/228,101

(22) Filed: Sep. 8, 2011

(51) Int. Cl. C10L 1/00; C11B 3/00; C12N 1/12

(52) U.S. Cl. 44/307; 435/257.1; 554/175

(57) ABSTRACT

A method is provided for growing microalgae from wastewater for oil production in a three-step wastewater treatment facility. In the method, two carbon sources are selected for addition to the wastewater, which contains naturally-occurring bacteria. Specifically, the first carbon source is selected to increase the carbon-to-nitrogen ratio and the carbon-to-phosphorous ratio within the microalgae. The first carbon source serves as a food source for the microalgae, and the second carbon source promotes the breakdown of carbon nitrogen and phosphorous by the bacteria cells into a more easily digestible form for the microalgae. Due to the added carbon, the wastewater supports growth of the microalgae and the production of oils therein.

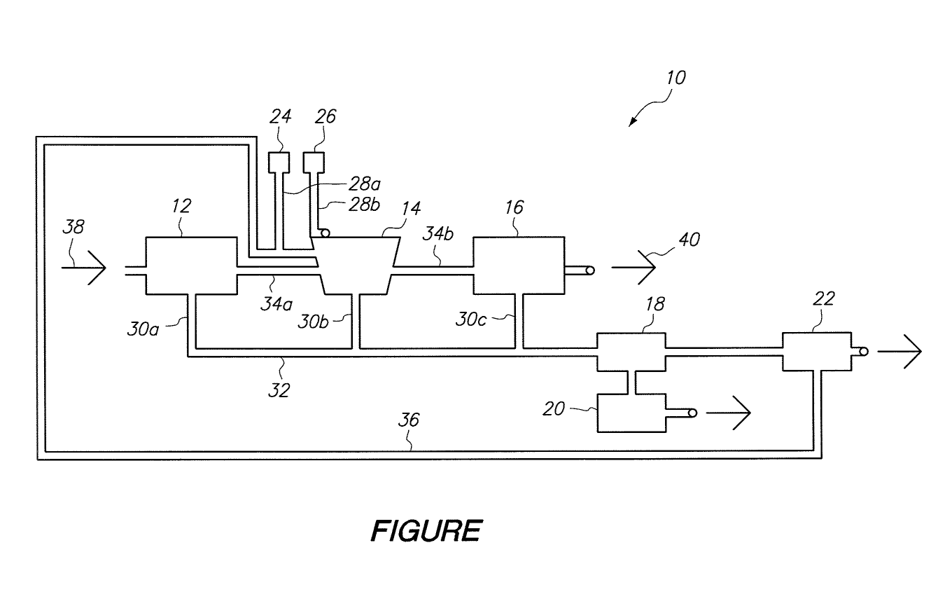

### FIELD OF THE INVENTION

The present invention pertains generally to methods for growing microalgae. More particularly, the present invention pertains to a method for growing microalgae in wastewater. The present invention is particularly, but not exclusively, useful as a method for enhancing microalgae growth in wastewater in order to produce biofuel more efficiently.

### BACKGROUND OF THE INVENTION

As worldwide petroleum deposits decrease, there is rising concern over petroleum shortages and the costs that are associated with the production of hydrocarbon products. As a result, alternatives to products that are currently processed from petroleum are being investigated. In this effort, biofuel, such as biodiesel, has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by maximizing the rate of growth of the plant source. Because microalgae is known to be one of the most efficient plants for converting solar energy into cell growth, it is of particular interest as a biofuel source. Importantly, the use of microalgae as a biofuel source presents no particularly exceptional problems, i.e., biofuel can be processed from oil in microalgae as easily as from oils in land-based plants.

While microalgae can efficiently transform solar energy into chemical energy via a high rate of cell growth, it has been difficult to create environments in which microalgae cell growth rates are optimized. Specifically, the conditions necessary to facilitate a fast growth rate for microalgae cells in large-scale operations have been found to be expensive to create. Further, certain conditions which may be ideal for microalgae growth may not be ideal for the growth of the types of microalgae that are most useful in the production of biofuels. For instance, wastewater treatment can benefit greatly from microalgae growth. However, such growth has generally been less than optimal for the production of oils for use in biofuels. Specifically, the species or strain of microalgae grown under such conditions is typically hard to control. In addition, the oil content resulting from microalgae grown in wastewater is often low.

In light of the above, it is an object of the present invention to provide a method for growing microalgae from wastewater for oil production. Another object of the present invention is to provide a method for preparing wastewater to support the growth of selected high oil microalgae strains. Another object of the present invention is to decrease costs by reducing the size of a facility required to use wastewater to produce microalgae that can be used to produce biofuel. Yet another object of the present invention is to provide a method for growing microalgae from wastewater for oil production that is simple to implement, easy to use, and comparatively cost effective.

### SUMMARY OF THE INVENTION

In the method of the present invention, wastewater is used to grow microalgae which can be used to produce biofuels. In the method of the present invention, two different carbon sources are added to promote efficient microalgae growth in wastewater. Once the microalgae have become rich in lipids (oil), the microalgae can be removed from the system and processed into biofuel.

Structurally, the present invention involves a wastewater treatment facility. A facility can be constructed to use the method of the present invention, or an existing facility can be adapted to use the method of the present invention. In the facility, a primary, a secondary, and a tertiary treatment apparatus are provided. Wastewater enters the facility at the primary treatment apparatus and exits the facility after passing through the tertiary treatment apparatus. In more detail, the primary treatment apparatus is any type that is commonly used in wastewater treatment for mechanical treatment of wastewater. And, in a preferred embodiment, the secondary treatment apparatus is a shallow pond. Connected to the secondary treatment apparatus are two reservoirs, one for holding microalgae inoculant and another for holding a carbon source. Additional structural components are provided to process the microalgae into biofuel. One is an oil extractor connected to the primary, secondary and tertiary treatment apparatuses. Another is a biomass digester connected to receive byproducts from the oil extractor. And, a biofuel reactor is connected to both the oil extractor and the secondary treatment apparatus. Using these three apparatuses, biomass, biogas, and liquid biofuel can be produced using the method of the present invention.

In operation, wastewater first undergoes conventional treatment at the primary treatment apparatus. Once primary treatment is completed, the wastewater is moved to the secondary treatment apparatus where microalgae and two different carbon sources are added to the wastewater. When determining the type of microalgae used, strains of microalgae are selected primarily based on the ability of the microalgae strains to use the selected carbon sources to produce lipids more rapidly. The first carbon source added to the wastewater is preferably CO₂. By adding CO₂, the microalgae have a primary food source and the carbon-to-nitrogen and carbon-to-phosphorous ratios are increased in the wastewater, while naturally occurring bacteria in the wastewater can break down the nitrogen and phosphorous into a simpler molecule that is more easily digestible by the microalgae cells. To promote the breakdown of the nitrogen and phosphorous by bacteria, another carbon source for the bacteria must be provided. For the present invention, this is done preferably by adding an organic carbon source, such as sugar, glycerin, citric acid, etc., to the secondary treatment apparatus to serve this purpose. Once the second carbon source is added, the bacteria can more effectively break down the nitrogen and phosphorous. In doing so, the microalgae will be able to more easily digest the nitrogen and phosphorous when in this form, and in turn, get into lipids (oil) formation phase at a much faster rate. By increasing the overall rate at which lipids are formed, shorter residence times are required for the microalgae in the secondary treatment step. And, shorter residence times mean increases in throughput rates which will increase the amount of biofuel produced by the system.

After the microalgae consume the nutrients in the secondary treatment step and become rich in lipids, they are removed from the wastewater for further processing. Upon removal of the microalgae, the wastewater undergoes a third treatment process. After completion of the third process, the wastewater is now treated water and it can be released from the system.

After the microalgae are removed from the secondary treatment apparatus, they enter into an oil extractor where lipids are removed. These lipids are then sent to a biofuel reactor for processing into liquid biofuel. Other byproducts removed during the first and third treatment steps are also fed into the oil extractor for the extraction of lipids which are sent to the biofuel reactor to produce biofuel. A byproduct of the biofuel production process is glycerin. Once removed from the system, the glycerin can be sent back to the secondary treatment apparatus for use as a carbon source for growing microalgae. In addition to biofuel, biogas may be produced by the system by removing biomass from the oil extractor and processing the biomass in a biomass digester.

### BRIEF DESCRIPTION OF THE DRAWINGS

The novel features of this invention, as well as the invention itself, both as to its structure and its operation, will be best understood from the accompanying drawing, taken in conjunction with the accompanying description, in which similar reference characters refer to similar parts, and in which the FIGURE is a schematic view of the method of the present invention.

### DESCRIPTION OF THE PREFERRED EMBODIMENTS

Referring to the FIGURE, an exemplary structural layout of the method for growing microalgae is illustrated. In the FIGURE, a wastewater treatment facility 10 is shown to include a primary treatment apparatus 12, a secondary treatment apparatus 14, and a tertiary treatment apparatus 16. More specifically, the secondary treatment apparatus 14 is a shallow treatment pond while the primary and tertiary treatment apparatuses are envisioned to be of any type commonly used in wastewater treatment. Additional structural components included in the wastewater treatment facility 10 for synthesizing biofuel include an oil extractor 18 connected to both a biomass digester 20 and a biofuel reactor 22. In addition to these components, two essential components are attached to the secondary treatment apparatus 14. These are a microalgae reservoir 24, and a carbon reservoir 26. Each of these components is connected to the secondary treatment apparatus 14 via a respective conduit 28a and 28b to allow for the addition of microalgae and carbon to the secondary treatment apparatus 14. In addition, as shown in the FIGURE, a drainage shaft 30a-c is connected to each of the three major treatment apparatuses (i.e. primary, secondary, and tertiary) to allow for the removal of byproducts of the wastewater treatment process into an oil extractor pipe 32 which feeds into the oil extractor 18. It can also be seen that transfer pipes 34a and 34b interconnect the three major treatment apparatuses, and a return pipe 36 runs between the biofuel reactor 22 and the secondary treatment apparatus 14.

Still referring to the FIGURE, an operation of the present invention can also be described. Wastewater enters the facility at the primary treatment apparatus 12 as indicated by arrow 38. Here, the wastewater undergoes a type of well-known mechanical treatment commonly used in the pertinent art. After undergoing primary treatment, the wastewater is piped through a transfer pipe 34a to the secondary treatment apparatus 14. At the same time, solid-based byproducts (mainly, sludge) travel through the drainage shaft 30a into the oil extractor pipe 32.

Once the wastewater reaches the secondary treatment apparatus 14, several actions occur. For one, microalgae cells from the microalgae reservoir 24 are released into the secondary treatment apparatus 14 to combine with the wastewater to form an effluent. Microalgae cells are continuously added to the secondary treatment apparatus 14 until equilibrium has been established between the microalgae growth rate and the rate of effluent leaving the secondary treatment apparatus 14. Once the effluent is formed, a second action occurs with CO₂ from the carbon reservoir 26 being added to the apparatus 14 and being absorbed by the microalgae cells to (1) serve as the primary carbon source for microalgae cell growth, and (2) increase the carbon-to-nitrogen and carbon-to-phosphorous ratios therein. As envisioned for the present invention, the CO₂ can be added by sparging or by a surface contact means. Importantly, the nitrogen and phosphorous present in the wastewater after primary treatment are in organic form, meaning the rate of absorption by the microalgae cells is slowed considerably. One mitigating consideration for the present invention is that naturally occurring bacteria in the wastewater can help breakdown the nitrogen and phosphorous into more easily absorbed molecules. But, this breakdown may be limited by carbon/energy sources available to the bacteria. In the present invention, a carbon feed is released from the carbon reservoir 26 into the secondary treatment apparatus 14 through the conduit 28b to promote the breakdown of nitrogen and phosphorous. This carbon feed may be sugar or any other carbon feed appropriate to promote growth of bacteria and, hence, the nitrogen and phosphorous breakdown by bacteria. When adding carbon feed, the amount added is limited to ensure that nearly all of the nitrogen and phosphorous nutrients are consumed by microalgae cells. Stated differently, the rate at which nitrogen and phosphorous are broken down should be about equal to the rate at which nitrogen and phosphorous cells are consumed by microalgae cells in the effluent. By ensuring consumption of nearly all nitrogen and phosphorous in the wastewater, nitrification and denitrification steps, which are common to wastewater treatment, can be eliminated. While these processes are occurring, the microalgae-rich effluent is removed through the drainage shaft 30b into the oil extractor pipe 32.

Once the processes in the secondary treatment apparatus 14 are completed, the wastewater travels through the transfer pipe 34b to the tertiary treatment apparatus 16. At this point, the wastewater undergoes any type of tertiary treatment well-known in the art before being released as treated water as indicated at arrow 40. Like with the primary and secondary treatment actions, any byproducts and residual effluent present in the tertiary treatment apparatus 16 will be drained through the drainage shaft 30c into the oil extractor pipe 32.

In order to form biofuel, byproducts and algal effluent pass from the three treatment apparatuses 12, 14, 16 into an oil extractor pipe 32. Once these materials reach the oil extractor 18, lipids are removed and sent to the biofuel reactor 22 to form liquid biofuel. During this process, a glycerin byproduct is formed at the biofuel reactor 22 and sent through the return pipe 36 to the secondary treatment apparatus 14 to be used as a food source for algal and bacteria cells. In addition to this production of liquid biofuel at the biofuel reactor 22, biogas production can also be a part of the method of the present invention. Specifically, a byproduct of the oil extractor 18 will be lean biomass. Instead of discarding the lean biomass, it can be sent to the biomass digester 20 to produce biogas.

While the particular Method for Growing Microalgae from Wastewater for Oil Production as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for growing microalgae from wastewater for biofuel production comprising the steps of:
transferring the wastewater from a primary treatment tank to a secondary treatment pond after a primary treatment, wherein the wastewater is phosphorous-rich (P) and nitrogen-rich (N) and contains naturally occurring bacteria;
inoculating the wastewater in the secondary treatment pond with a selected strain of microalgae cells to create an effluent;
introducing carbon dioxide (CO₂) into the secondary treatment pond for assimilation by the microalgae cells in the effluent to increase the C:N ratio and C:P ratio within the microalgae cells;
selecting a carbon feed to be added to the effluent in the secondary treatment pond; and
adding the selected carbon feed to the effluent in the secondary treatment pond to increase the consumption of phosphorous and nitrogen by the microalgae cells.

2. A method as recited in claim 1 wherein, during the introducing step, the selected carbon feed is digestible only by the selected strains of microalgae cells.

3. A method as recited in claim 1 wherein the selected strain of microalgae cells are selected to promote the production of microalgae with a higher oil content.

4. A method as recited in claim 1 further comprising the steps of:
removing microalgae cells with a high lipid content from the effluent;
transferring the removed microalgae cells to an oil extractor to extract lipids from the removed microalgae cells; and
transferring the lipids to a biofuel reactor to produce liquid biofuel and glycerin.

5. A method as recited in claim 4 wherein the glycerin produced at the biofuel reactor is added to the secondary treatment pond.

6. A method as recited in claim 1 wherein the carbon feed is selected from a group comprising sugar, glycerin and citric acid.

7. A method as recited in claim 1 wherein the carbon feed is cellulose.

8. A method as recited in claim 1 wherein the inoculating step is completed when an equilibrium is established between a microalgae growth rate and an effluent rate of wastewater being removed from the secondary treatment pond.

9. A method as recited in claim 1 further comprising the step of transferring the wastewater to a tertiary treatment device after the wastewater leaves the secondary treatment pond.

10. A method for growing microalgae from wastewater for biofuel production comprising the steps of:
selecting a first carbon source for use by a chosen strain of microalgae, wherein the chosen strain of microalgae is comprised of a plurality of microalgae cells;
inoculating the wastewater with the chosen strain of microalgae, to form an effluent of the chosen strain of microalgae, wherein the wastewater contains bacteria cells;
adding the first carbon source to the wastewater to increase the ‘ carbon-to-nitrogen ratio and the carbon-to-phosphorous ratio in the microalgae cells; and
introducing a second carbon source into the wastewater to promote nitrogen and phosphorous breakdown processes by the bacteria cells in the wastewater.

11. A method as recited in claim 10 wherein the first carbon source is carbon dioxide.

12. A method as recited in claim 10 wherein the second carbon source is cellulose.

13. A method as recited in claim 10 wherein the second carbon source is selected from a group comprising sugar, glycerin and citric acid.

14. A method as recited in claim 10 wherein the first carbon source can only be digested by a group of microalgae strains including the chosen microalgae strain.

15. A method as recited in claim 10 wherein the wastewater is provided after a primary wastewater treatment process.

16. A method as recited in claim 10 further comprising the steps of:
transferring the effluent to an oil extractor to extract lipids from the microalgae cells; and
transferring the wastewater to a tertiary treatment device.

17. A method for growing microalgae from wastewater for biofuel production comprising the steps of:
selectively adding carbon dioxide to the wastewater to favor microalgae growth over bacterial growth, wherein adding carbon dioxide increases the carbon-to-nitrogen ratio and the carbon-to-phosphorous ratio within the microalgae;
selectively adding a carbon source to the wastewater to promote the breakdown of nitrogen and phosphorous by bacteria cells present in the wastewater; and
inoculating the wastewater with microalgae, with the wastewater supporting growth of the microalgae and the production of oils therein.

18. A method as recited in claim 17 wherein the wastewater does not contain any of the selected microalgae strains before the inoculating step.

19. A method as recited in claim 17 wherein the wastewater contains one or more microalgae strains before the inoculating step.

20. A method as recited in claim 17 wherein carbon dioxide is replaced by an alternate carbon source to favor microalgae growth over bacterial growth.
